(19) United States
(12) Patent Application Publication
(10) Pub. No.: US 2012/0258083 A1
(43) Pub. Date: Oct. 11, 2012

(54) PLACENTAL VASCULAR LOBULE STEM CELLS

(76) Inventor: Michael P. Murphy, Carmel, IN (US)

(21) Appl. No.: 13/527,579

(22) Filed: Jun. 19, 2012

Related U.S. Application Data
(63) Continuation of application No. 12/355,682, filed on Jan. 16, 2009.
(60) Provisional application No. 61/021,592, filed on Jan. 16, 2008.

(51) Int. Cl. A61K 35/50; C12N 5/071; A61P 9/10

(52) U.S. Cl. 424/93.7; 424/583; 435/325

(57) ABSTRACT

The present invention provides isolated populations of stem and progenitor cells from fetal vascular lobules of the placenta. The isolated populations of stem and progenitor cells of the invention express the markers CD144, CD105, and/or CD31 and lack expression of the hematopoietic-lineage marker CD45. Under specific conditions, cells of the invention may function as endothelial precursors and may provide therapeutic preparations, for example, in the treatment of ischemia.

### RELATED APPLICATIONS

This application claims the benefit of priority under 35 USC §119 of U.S. Provisional Application Ser. No. 61/021,592 filed Jan. 16, 2008, the entire disclosure of which is incorporated herein by reference.

### FIELD OF THE INVENTION

The invention relates to the field of cell biology, cell culture, and regenerative medicine. In particular, the invention relates to the area of cellular therapies, more specifically, the invention relates to cellular populations and products thereof derived from the placenta that are useful for regenerative applications.

### BACKGROUND

Endothelial progenitor cells have been the examined both as a biomarker of disease as well as a source of cell therapies to treat cardiovascular disorders. Hallmarks of a stem/progenitor population are the ability for self renewal and the capacity to terminally differentiate into a mature phenotype. Although some clinical evaluation has been performed using autologous adult stem cells, these stem cell possess a lower angiogenic ability as compared to endothelial progenitors found in younger tissue such as cord blood. Previously it has been reported that cord blood possesses endothelial progenitor cells, however such cells have not been described in the tissue component of the placenta. The current invention describes a novel stem cell population derived from the fetal vascular lobules of the hemochorial placenta.

### SUMMARY OF THE INVENTION

The present invention provides isolated stem cell and endothelial progenitor cell populations derived from fetal vascular lobules of a hemochorial placenta, particularly a hemochorial plancenta from a human. In one embodiment of the invention the stem and/or endothelial progenitor cells express CD144, CD105, and/or CD31, either immediately upon isolation or after culturing. In certain aspects, stem cells and/or endothelial progenitor cells of the invention do not express CD45. In one embodiment of the invention, the stem and/or endothelial progenitor cells express CD144, CD105, and CD31 but do not express do not express CD45.

Certain isolated stem cell and endothelial progenitor cell populations of the invention can form capillary-like tubules when plated on a Matrigel substrate and can take up DiI-acetylated-low-density-lipoprotein.

In certain embodiments, the isolated stem and/or endothelial progenitor cell populations of the invention are prepared by homogenizing fetal vascular lobules from a full-term placenta; successively digesting the homogenized lobules with a preparation of about 2% collagenase, about 0.25% trypsin and about 0.1% DNAse, in tissue culture medium such as DMEM. The digestion product is then filtered to remove particulates, and mononuclear cells are obtained therefrom by density gradient centrifugation. The mononuclear cells can then be plated on collagen I-coated tissue culture plates and grown to confluency. Detached cells from the confluent plates are then sorted to obtain stem and/or progenitor cells that express of CD144 but lack of expression of CD45.

In certain embodiments of the invention, the isolated stem and/or endothelial progenitor cells of the invention have the ability to differentiate into mesoderm, ectoderm and endoderm. In other embodiments of the invention, the isolated stem and/or endothelial progenitor cells of the invention have the ability to differentiate into at least one of mesoderm, ectoderm or endoderm.

The present invention also provides methods for treating ischemic disorders in a subject. In one embodiment, this method involves identifying an area of reduced blood flow in a subject and administering isolated stem or endothelial progenitor cells isolated as described above to an area proximal to the area of reduced blood flow.

In certain aspects, the isolated stem or endothelial progenitor cells can be expanded in vitro prior to administration. In other embodiments, the isolated stem or endothelial progenitor cells can be activated in vitro prior to administration. Activation can, for example, include treatment with at least one agent or condition capable of upregulating a CXCR-4 receptor, such as IL-1, IL-6, stem cell factor, flt-3L, hepatocyte growth factor, exposure to hypoxic conditions, a cytokine, a histone deacetylating agent, a DNA methyltransferase inhibitor, and an inhibitor of GSK-3 kinase.

Such activated cells may have, for example, enhanced migration towards ischemic tissue as compared to unactivated stem cells; enhanced proliferative ability as compared to unactivated stem cells; enhanced differentiation ability as compared to unactivated stem cells; enhanced growth factor secretion activity as compared to unactivated stem cells; enhanced angiogenic activity as compared to unactivated stem cells; and enhanced ability to stimulate proliferation and/or mobilization of endogenous stem cells as compared to unactivated stem cells.

Also provided by the present invention are therapeutic compositions including culture supernatant of an isolated stem cell population or an isolated endothelial progenitor cell population derived from fetal vascular lobules from hemochorial placenta. The supernatant can be prepared by culture of stem or progenitor cells of the invention in a culture medium suitable for maintain viability of the stem cells for a period of time sufficient for the stem cells to secret therapeutic factors into the culture medium. Such therapeutic factors include, but are not limited to, growth factors, anti-apoptotic agents, factors that stimulate proliferation of endogenous stem cells, angiogenic factors, and factors capable of mobilizing stem cells. Furthermore, culture of the stem and/or progenitor cells can be performed under hypoxic conditions; with administration of cytokines or epigenetically-acting agents; or by genetic manipulation in order to stimulate a biological property of the cells.

The present invention also provides therapeutic compositions including isolated stem and/or progenitor cells derived from fetal lobules of a hemochorial placenta and a pharmaceutically applicable medium suitable for administration to a subject.

### BRIEF DESCRIPTION OF THE DRAWINGS

FIG. 1 displays pictures of placental lobes at different stages of processing.

FIG. 1A shows the placental lobe after fresh isolation; while FIG. 1B shows the same lobe after gently removing the decidual tissue exposing the rich capillary network in the lobe.

FIG. 2A displays fluorescence activated cell sorting result of monolayers derived from colonies. CD45 depleted CD144 positive cells were collected as ECFCs.

FIG. 2B Fluorescence cytometry analysis of placenta derived ECFCs (PDECFCs). The cells express CD31, CD 144, CD105, KDR, but do not express CD45 and CD34. Isotype controls are overlaid in black line on each histogram for each surface antigen tested. The result is representative data from 15 different placentas with similar results.

FIG. 3A displays PECFCs plated in Matrigel for the formation of capillary-like structures. The pictures were taken after 24 hour incubation of cells on Matrigel.

FIG. 3B PECFCs incorporate Dil-Ac-LDL (50× magnification). A representative microscopic view is shown for PDECFCs, which shows that the ECFCs have taken up Dil-Ac-LDL (red) and have nuclei stained with DAPI (blue).

FIG. 4 displays quantitation of the clonogenic and proliferative potential of single placenta derived ECFC. Microscopic views (1) to (6) of the different PDECFC clusters (less than 50 cells) or colonies (more than 50 cells) derived from single PECFC. Similar results were observed in 4 different experiments using different PECFCs.

FIG. 5 displays, number of cell progenies derived from a single PECFC compared to cord blood ECFCs in individual wells 14 days post-culture. Results represent the average +/− SEM of four independent experiments.

FIG. 6 displays growth kinetics of PECFCs. Cells were quantified over 24 passages and the number of cells were plotted against the days in culture. This graph is comparable to growth kinetics curves previously established for other sources of ECFCs.

### DETAILED DESCRIPTION OF THE INVENTION

It is to be understood that both the foregoing general description and the following detailed description are exemplary and explanatory only and are not restrictive of the invention, as claimed. Moreover, it must be understood that the invention is not limited to the particular embodiments described, as such may, of course, vary. Further, the terminology used to describe particular embodiments is not intended to be limiting, since the scope of the present invention will be limited only by its claims.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention. Further, all publications mentioned herein are incorporated by reference.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. It must be noted that, as used herein and in the appended claims, the singular forms “a,” “or,” and “the” include plural referents unless the context clearly dictates otherwise. Thus, for example, reference to “a stem cell” includes a plurality of such stem cells and reference to “the agent” includes reference to one or more agents and equivalents thereof known to those skilled in the art, and so forth.

“About” as used herein means that a number referred to as “about” comprises the recited number plus or minus 1-10% of that recited number. For example, “about” 100 percent can mean.95-105 percent or as few as 99-101 Perecent depending on the situation. Whenever it appears herein, a numerical range such as “1 to 20” refers to each integer in the given range; e.g., “a population of 1 to 20 cells” means that the population group can contain only 1 cell, 2 cells, 3 cells; etc., up to and including 20 cells although the term “population” also includes instances where no numerical range is designated.

Further, all numbers expressing quantities of ingredients, reaction conditions, % purity, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

Standard techniques may be used for recombinant DNA, oligonucleotide synthesis, and tissue culture and transformation (e.g., electroporation, lipofection, etc.). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients.

The terms “patient,” “subject,” and “individual,” are used interchangeably herein, to refer to mammals, including, but not limited to, humans, murines, simians, felines, canines, equines, bovines, porcines, ovines, caprines, avians, mammalian farm and agricultural animals, mammalian sport animals, and mammalian pets. In certain embodiments of the invention, the subject is a human patient.

The following examples, including the experiments conducted and results achieved are provided for illustrative purposes only and are not to be construed as limiting the present invention in any way.

The present invention provides novel isolated populations of stem cells and endothelial progenitor cells prepared from fetal vascular lobules of the placenta. The cell populations of the invention express the markers CD144, CD105, and CD31 and lack expression of the hematopoietic-lineage marker CD45. Under specific conditions, isolated cell populations of the invention may function as endothelial precursor cells given their ability to form capillary-like tubules and uptake DiI-acetylated-low-density-lipoprotein. In terms of endothelial colony formation, fetal vascular lobule-derived stem cells are superior to stem cells isolated from the umbilical cord blood. In certain aspects, fetal vascular lobule-derived stem cells of the invention are capable of proliferating up to 46 doublings before senescence. In other aspects, the fetal vascular lobule-derived stem cells of the invention are capable of proliferating for up to at least 30, 35, 40, or 45 doublings before senescence. In other aspects of the invention stem cell properties of the fetal vascular lobule derived stem cells, supernatants, or isolated factors contain in such supernatants promote and can be used to stimulate tissue regeneration, repair, provide trophic support, anti-apoptotic function or radioprotection in a patient in need thereof.

The present invention provides an isolated population of cells derived from the fetal vascular lobes of the hemochorial placenta, e.g., of a human subject. In one aspect of the invention, the isolated population of cells includes stem cells. In another aspect of the invention, the isolated population of cells includes progenitor cells.

In certain embodiments, the isolated population of cells of the invention express CD144, CD105, and CD31, and do not express the marker CD45. Isolated populations of stem and progenitor cells of the invention have therapeutic properties and can be administered to a subject e.g., to stimulate tissue regeneration, repair, trophic support, anti-apoptotic function or radioprotection.

The present invention also provides therapeutic compositions comprising supernatant of stem and progenitor cells of the invention, which may be administered alone or in combination with other therapies.

In yet another aspect of the invention, a method of treating an ischemic disease is provided through the administration of cells derived from fetal vascular lobules that express the markers CD144, CD105, and CD31, and do not express the marker CD45.

### EXAMPLES

### Example 1

### Isolation and Characterization of Fetal Vascular Lobule Derived Stem Cells from Hemochorial Placenta

Placenta Collection:

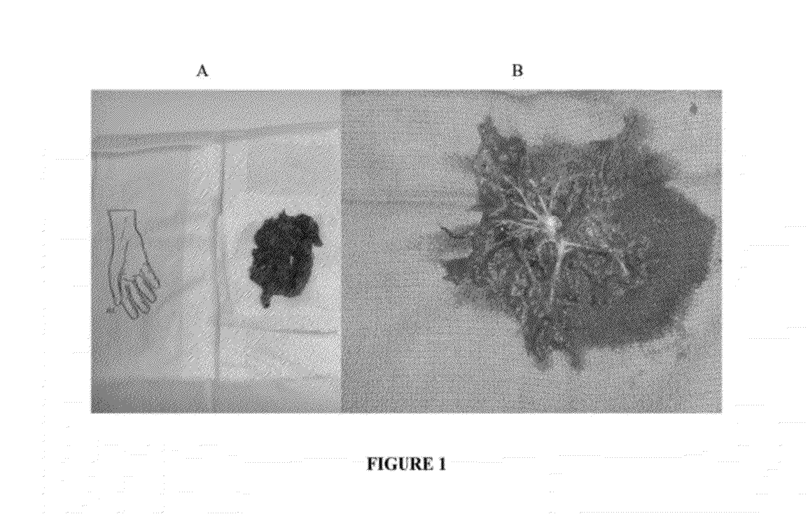

With Approval from the IRB of Wishard Health Systems and informed consent, full term placentas were obtained from cesarean sections. Fetal vascular lobules (FIG. 1) were isolated from the placental tissue and placed in HBSS.

Cell Isolation and Culture:

Fetal vascular lobules were placed in a blender with HBSS and homogenized. The homogenate was centrifuged at 600×g for 6 minutes and washed three times with PBS. The pelleted cells were then digested with 2% collagenase in DMEM, 0.25% trypsin and 0.1% DNase in sequence. The resulting preparation was filtered and the mononuclear cell fraction (MNC) was isolated with Ficoll gradient centrifugation and plated on six-well plates pre-coated with collagen type I at 50 million cells/well.

Flow Cytometric Characterization of Cells:

Cells were grown to confluency, detached and sorted using a fluorescent activated cell (FACS) Aria Sorter with antibodies to CD45 and CD144. CD144⁺/CD45⁻ cells were replated on type 1 rat tail collagen and the media changed every 48 hours. At 2-3 passages the cell surface markers were analyzed with FACS for CD31, CD105, CD45, CD144, CD34, and KDR.

Population Doubling:

Cells were enumerated at first passage and each subsequent passage for calculation of population doubling times (PDT). The PDT was derived using the time interval between cell seeding and harvest divided by the number of PDs for that passage.

Single Cell Clonogenic Assay:

One CD144⁺/CD45⁻ cell with 200 ml of EGM2 was placed in each well of a 96 well tissue culture plated coated with type 1 rat tail collagen. On day 14, wells containing greater than 50 cells (as determined under light microscopy at 40×) were considered colonies, trypsinized and counted with a hemocytometer. PECFC colonies were compared to cord blood ECFC colonies from the same patient.

Matrigel Tube Formation:

5×10³ cells (passage 3-4) were plated in each of three wells containing Matrigel basement membrane matrix. At 24 hours, four representative fields were taken and the number of complete tubes formed by cells per well was counted.

Uptake of Acetvlated Low-Density-Lipoprotein:

Cells were incubated for 24 hours with 10 μg/ml of Dil-Ac-LDL in EGM2 and observed under the fluorescent microscope for evidence of Dil-Ac-LDL uptake.

### Results

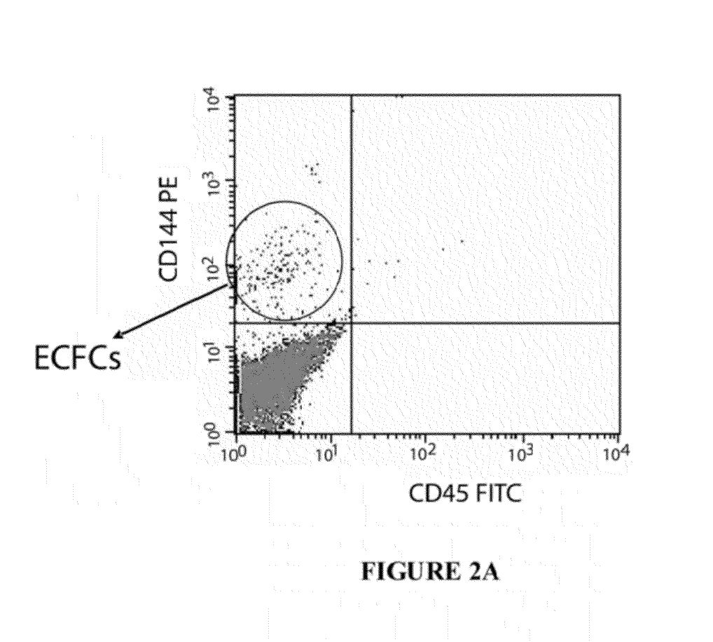

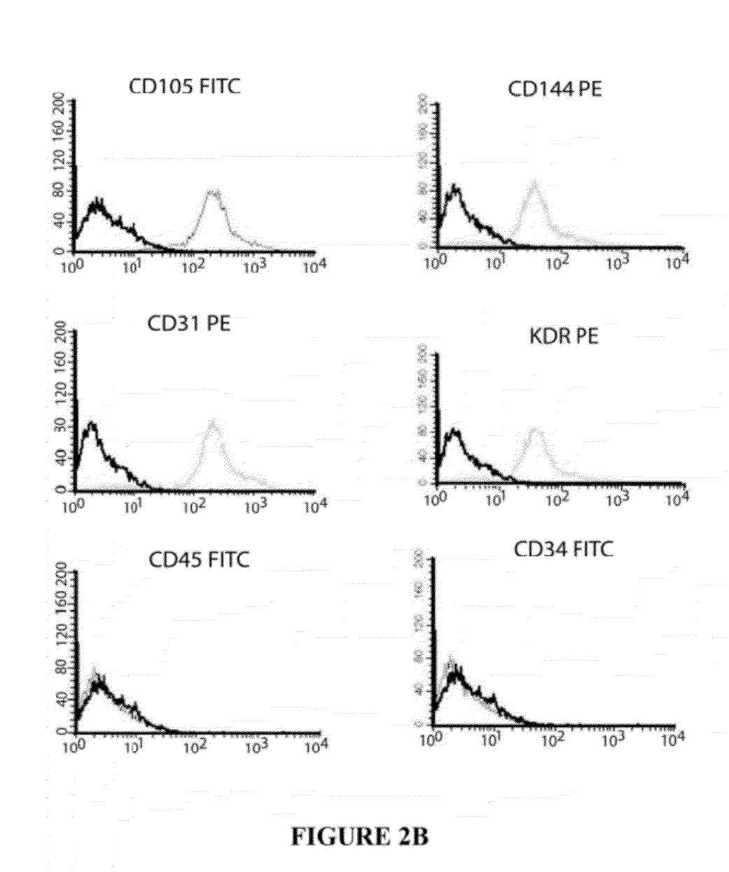

Using the culture techniques described by Ingram for the isolation of ECFCs from umbilical cord blood, cells isolated from fetal placental vascular lobules were positive for CD144, CD105, and CD31 and were negative for CD45 consistent with the phenotypic description of ECFCs (FIG. 2).

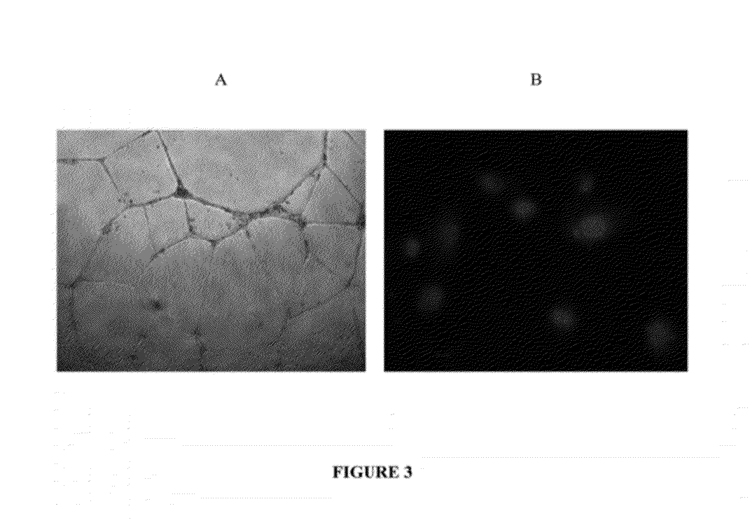

In addition, placental ECFCs (PECFCs) formed capillary-like tubes in vitro and were found to take-up DiI-acetylated-low-density-lipoprotein (FIG. 3).

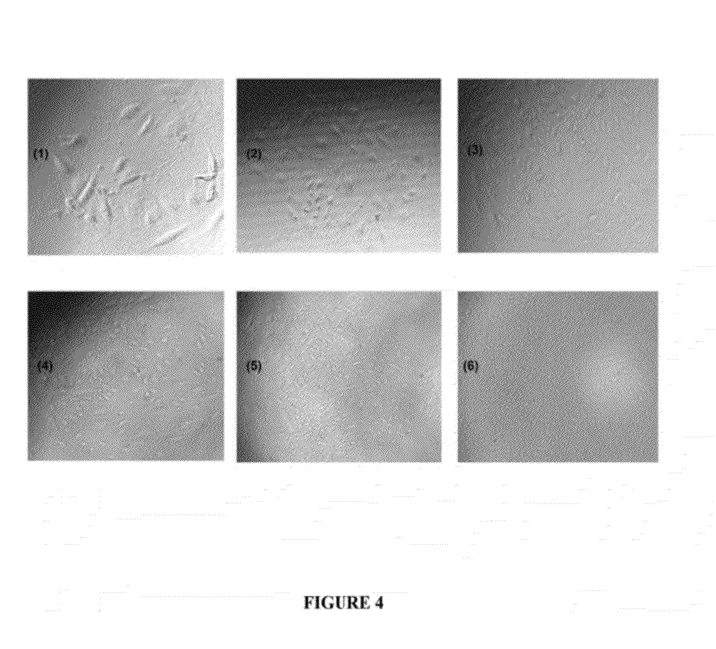

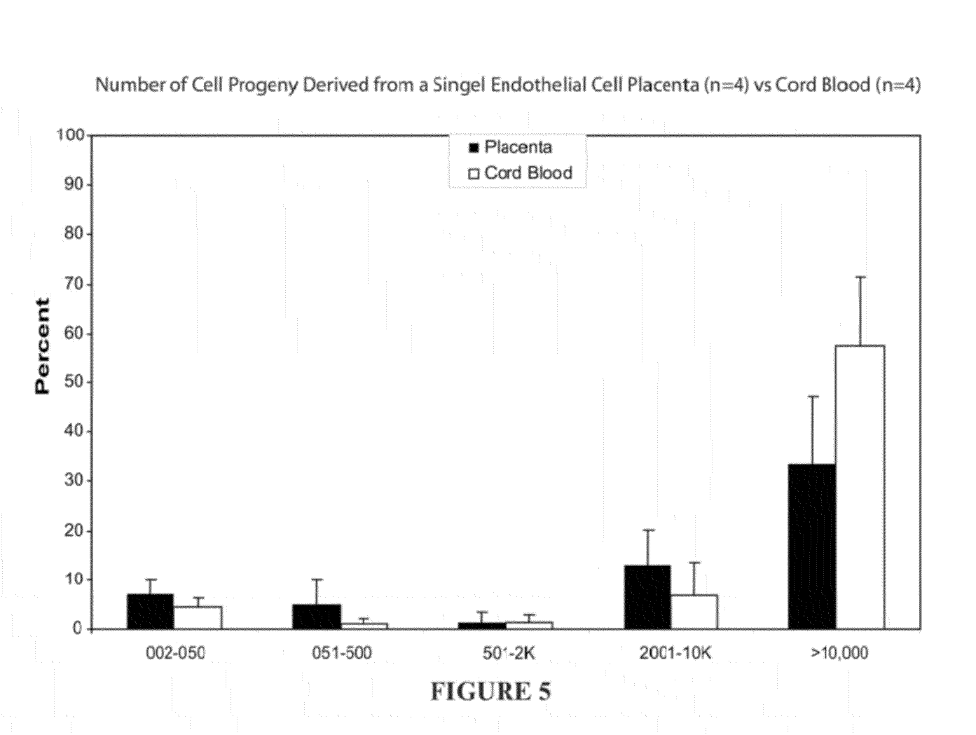

The single cell clonogenic assay demonstrated that colonies of PECFCs will form from a single PECFC (FIG. 4).

A single PECFC exhibited similar colony forming potential compared to a single cord blood ECFC (FIG. 5).

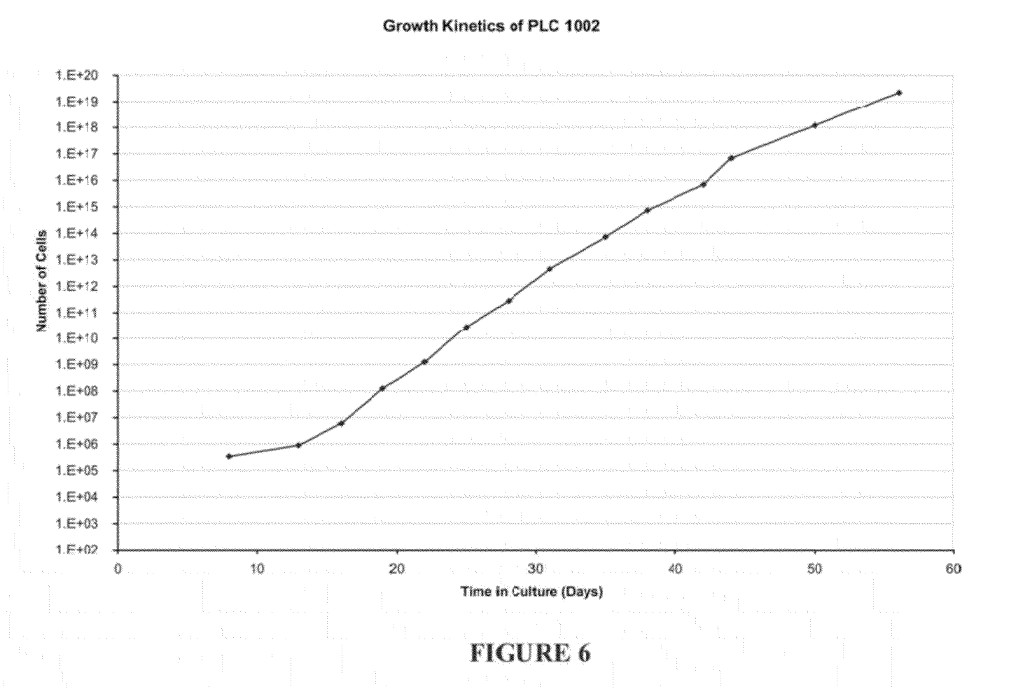

Additionally, when cultured in a T75 flask, placental ECFCs grew at a remarkable rate, and reached 46 population doublings before becoming senescent (FIG. 6).

Placental tissue was found to be a rich source of ECFCs, which exhibited extraordinary proliferative potential, as demonstrated by the single cell clonogenic assay. The highly proliferative PECFCs were similar to highly proliferative cord blood derived ECFCs with respect to growth kinetics, morphology, surface marker expression and tube formation. Because of the immunotolerant properties of the placenta, these cells can be used as a source of allogeneic cell-based therapies.

1-28. (canceled)

29. A method for isolating stem cells or endothelial progenitor cells comprising obtaining CD144⁺CD45⁻ mononuclear cells from fetal vascular lobules of full-term placenta.

30. The method according to claim 29, further comprising:
a) homogenizing fetal vascular lobules from a full-term placenta;
b) successively digesting the homogenized lobules of step a) with a preparation of about 2% collagenase, about 0.25% trypsin and about 0.1% DNAse in tissue culture medium;
c) filtering the digestion product of step b) to remove particulates;
d) obtaining a mononuclear cells from the filtered digestion product of step c) by density gradient centrifugation;
e) plating the mononuclear cells on a collagen I-coated tissue culture plate;
f) growing the mononuclear cells to confluency;
g) detaching the confluent cells from the plate; and
h) sorting the detached cells for expression of CD144 and lack of expression of CD45.

31. The method according to claim 30 further comprising sorting the detached cells for expression of CD31.

32. The method according to claim 30 further comprising sorting the detached cells for expression of CD105.

33. An isolated stem cell population obtained by the method according to claim 29.

34. The isolated stem cell population of claim 33, wherein said stem cells further express at least one marker selected from: CD105 and CD31.

35. The isolated stem cell population of claim 33, wherein said cells form capillary-like tubules when plated on a Matrigel substrate.

36. The isolated stem cell population of claim 33, wherein said cells take up DiI-acetylated-low-density-lipoprotein.

37. The isolated stem cell population of claim 33, wherein said cells have the ability to differentiate into mesoderm, ectoderm and endoderm.

38. An isolated population of endothelial progenitor cells obtained by the method according to claim 29.

39. The isolated population of endothelial progenitor cells of claim 38, wherein said endothelial progenitor cells express at least one marker selected from: CD105 and CD31.

40. The isolated endothelial progenitor population of claim 38, wherein said endothelial progenitors cells form capillary-like tubules when plated on a Matrigel substrate.

41. The isolated endothelial progenitor population 38, wherein said endothelial progenitors take up DiI-acetylated-low-density-lipoprotein.

42. A method of treating an ischemic disorder comprising the steps of:
a) isolating a population of stem cells according to the method of claim 29;
b) identifying an area of reduced blood flow in a subject; and
c) administering the stem cells of step a) to an area proximal to the area of reduced blood flow of step b).

43. The method according to claim 42, wherein the stem cells are prepared according to the steps of:
a) homogenizing fetal vascular lobules from a full-term placenta;
b) successively digesting the homogenized lobules of step a) with a preparation of about 2% collagenase, about 0.25% trypsin and about 0.1% DNAse in tissue culture medium;
c) filtering the digestion product of step b) to remove particulates;
d) obtaining a mononuclear cells from the filtered digestion product of step c) by density gradient centrifugation;
e) plating the mononuclear cells on a collagen I-coated tissue culture plate;
f) growing the mononuclear cells to confluency;
g) detaching the confluent cells from the plate; and
h) sorting the detached cells for expression of CD144 and lack of expression of CD45.

44. The method of claim 43, further comprising expanding the sorted cells of step h) in vitro.

45. The method of claim 44, further comprising activating the stem cells in vitro.

46. The method of claim 45, wherein the activated stem cells of claim 17 have at least one property selected from: enhanced migration towards ischemic tissue as compared to unactivated stem cells; enhanced proliferative ability as compared to unactivated stem cells; enhanced differentiation ability as compared to unactivated stem cells; enhanced growth factor secretion activity as compared to unactivated stem cells; enhanced angiogenic activity as compared to unactivated stem cells; and enhanced ability to stimulate proliferation and/or mobilization of endogenous stem cells as compared to unactivated stem cells.

47. The method of claim 45, wherein the activating of the stem cells in vitro comprises treatment with at least one agent or condition capable of upregulating a CXCR-4 receptor.

48. The method of claim 47, where the agent or condition capable of upregulating a CXCR-4 receptor is selected from: IL-1, IL-6, stem cell factor, flt-3L, hepatocyte growth factor, exposure to hypoxic conditions, a cytokine, a histone deacetylating agent, a DNA methyltransferase inhibitor, and an inhibitor of GSK-3 kinase.

49. A therapeutic composition comprising the culture supernatant of the isolated stem cell population of claim 35.

50. The composition of claim 49, wherein said supernatant is generated by culture of said stem cells in a culture medium suitable for maintain viability of the stem cells for a period of time sufficient for the stem cells to secret therapeutic factors into the culture medium.

51. The composition of claim 50, wherein said therapeutic factors are selected from: growth factors, anti-apoptotic agents, factors that stimulate proliferation of endogenous stem cells, angiogenic factors, and factors capable of mobilizing stem cells.

52. The composition of claim 50, wherein culture of said stem cells comprises at least one of: hypoxia; administration of cytokines; administration of epigenetically-acting agents; and
genetic manipulation; and thereby stimulates a biological property of the stem cells.

53. A therapeutic composition comprising the isolated stem cells of claim 35 and a pharmaceutically applicable medium suitable for administration to a subject.
